(19) United States
(12) Patent Application Publication
(10) Pub. No.: US 2025/0221640 A1
(43) Pub. Date: Jul. 10, 2025

(54) COMMUNICATION SYSTEM OF ANALYTE DETECTION DEVICE

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/695,364

(22) PCT Filed: Aug. 1, 2022
(86) PCT No.: PCT/CN2022/109439

§ 371 Date: Mar. 26, 2024

(30) Foreign Application Priority Data
Sep. 27, 2021 (WO) PCT/CN2021/120856
Jun. 17, 2022 (WO) PCT/CN2022/099387

(51) Int. Cl. A61B 5/145; G16H 40/67

(52) U.S. Cl. CPC A61B 5/14546; G16H 40/67; A61B 2560/0209

(57) ABSTRACT

The invention discloses the communication system of analyte detection device, which comprises the analyte detection device and the remote equipment. The analyte detection device transmits signals, and the remote equipment is used to search and identify nearby signals, establish the communication connection with the analyte detection device and perform data interaction. The data interaction mode can be adjusted according to the actual use situation, and the battery energy consumption is reduced while transmitting the analyte parameter information, it is conducive to the long-time work of the system and improves the user experience.

### TECHNICAL FIELD

The invention mainly relates to the field of medical devices, in particular to a communication system of analyte detection device.

### BACKGROUND

The pancreas in a normal human body can automatically monitor the blood glucose level and automatically secrete required amount of insulin/glucagon. In the body of a type 1 diabetes patient, the pancreas does not function properly and cannot produce enough insulin for the body. Therefore, type 1 diabetes is a metabolic disease caused by abnormal pancreatic function, and diabetes is a lifelong disease. At present, there is no cure for diabetes with medical technology. The onset and development of diabetes and its complications can only be controlled by stabilizing blood glucose.

Diabetics need to have their blood glucose measured before they inject insulin into the body. At present, most of the testing methods can continuously measure blood glucose level and transmit the data to a remote device in real time for the user to view. This method is called Continuous Glucose Monitoring (CGM).

After the analyte detection device of the prior art establishes communication connection with the remote equipment, the higher power is used to transmit signals, so that the remote equipment can maintain the communication connection with the analyte detection device. However, this communication connection mode requires more energy consumption of the battery, which is not conducive to the long-term work of the system.

Therefore, the existing technology urgently needs the communication system of analyte detection device with low power consumption.

### BRIEF SUMMARY OF THE INVENTION

The invention discloses a communication system of analyte detection device, which comprises the analyte detection device and the remote equipment. The analyte detection device transmits signals, and the remote equipment is used to search and identify nearby signals, establish the communication connection with the analyte detection device and perform data interaction. The data interaction mode can be adjusted according to the actual use situation, and the battery energy consumption is reduced while transmitting the analyte parameter information, it is conducive to the long-time work of the system and improves the user experience.

The embodiment of the invention discloses a communication system of analyte detection device, comprising: the analyte detection device, which is installed on the surface of the user's skin, obtains the analyte parameter information in the user's body, and transmits signals to the remote equipment; And the remote equipment, which is used to search and identify nearby signals, establish communication connection with analyte detection device, and execute data interaction program, which at least includes scanning program and real-time program. Wherein, when the scanning program is executed, the analyte detection device transmits the scanning signal with the first power, when the real-time program is executed, the analyte detection device transmits the real-time signal with the second power, and the first power is lower than the second power.

According to one aspect of the invention, the first power and the second power are adjusted according to the communication distance of the signal to adjust the effective distance of the scanning signal or the real-time signal.

According to one aspect of the invention, the effective distance of the scanning signal is 0˜1 m, and the effective distance of the real-time signal is 0˜10 m.

According to one aspect of the invention, when the remote equipment is within the effective distance, the remote equipment establishes the communication connection with the analyte detection device, otherwise, the communication connection is not established.

According to one aspect of the invention, when the scanning program is executed, the analyte detection device sends the analyte parameter information to the remote equipment at least once.

According to one aspect of the invention, the analyte parameter information includes real-time analyte parameter information and/or historical analyte parameter information.

According to one aspect of the invention, when the real-time program is executed, the analyte detection device sends real-time analyte parameter information to the remote equipment at a fixed time interval.

According to one aspect of the invention, the difference between the scanning signal and the real-time signal also lies at least in the signal frequency, signal type or/and signal format.

According to one aspect of the invention, before the communication connection between the remote equipment and the analyte detection device is established, the remote equipment sends a prompt that needs to be confirmed by the user.

According to one aspect of the invention, after the remote equipment establishes the the communication connection with the analyte detection device, the analyte detection device is stored as the white list device of the remote equipment, and the remote equipment only receives the signal transmitted by the white list device.

According to one aspect of the invention, the scanning program and the real-time program are switched by the control command.

According to one aspect of the invention, after the remote equipment searches for the signal, before establishing communication with the analyte detection device, the remote equipment and the analyte detection device execute the communication data interaction program, and the preset effective time of the communication data interaction program is t.

According to one aspect of the invention, the effective time t of the communication data interaction program is 2˜7 s.

According to one aspect of the present invention, the remote equipment is configured so that when the communication data interaction program time T is shorter or longer than the effective time t, the remote equipment does not establish the communication connection with the analyte detection device.

Compared with the prior art, the technical scheme of the invention has the following advantages:

In the communication system of the analyte detection device disclosed by the invention, the data interaction mode between the analyte detection device and remote equipment can be adjusted according to the actual use situation. While transmitting the analyte parameter information, the battery energy consumption is reduced, which is conducive to the long-time work of the system and improves the user experience.

Further, users can use different data interaction modes according to different use scenarios to obtain the analyte parameter information required by users, which improves the user experience.

Further, the signal transmission power of the analyte detection device can be adjusted according to the communication distance to adjust the effective distance of the signal, so that the remote equipment can receive high-quality signals, but also reduce the battery energy consumption, which is conducive to the long-term work of the system and improve the user experience.

Further, the power of the scanning signal is lower than the power of the real-time signal, that is, the effective distance of the scanning signal is less than the effective distance of the real-time signal, which conforms to the application scenarios of the two signals. At the same time, it can avoid the interference between the two signals to ensure the signal quality.

Furthermore, the difference between the scanning signal and the real-time signal lies at least in the signal frequency, signal type or/and signal format. The two signals can be further distinguished to avoid interference between the two signals, so as to ensure the signal quality.

Further, before the communication connection between the remote equipment and the analyte detection device is established, the remote equipment sends a prompt that needs to be confirmed by the user, so as to prevent the remote equipment from establishing the communication connection with the analyte detection device at the wrong time, or connecting to the wrong analyte detection device, which improves the user experience.

Further, a white list is set in the remote equipment, and the analyte detection device that establishes the communication connection with the remote equipment is stored as the white list equipment. The remote equipment only accepts the signals transmitted by the white list equipment, so the remote equipment can only re-establish the communication connection with the white list equipment, or can only receive the analyte parameter information from the white list equipment, avoiding the wrong signal interference and improving the reliability of the signal.

### BRIEF DESCRIPTION OF THE DRAWINGS

FIG. 1 is a schematic diagram of the structure of the analyte detection system according to an embodiment of the invention;

FIG. 2 is a schematic diagram of the analyte detection device according to an embodiment of the invention;

FIG. 3a is a structural schematic diagram of the wake-up module of the analyte detection device comprising a sensor according to an embodiment of the invention;

FIG. 3b is a schematic diagram of the wake-up module of the analyte detection device comprising a light sensing element according to an embodiment of the invention;

FIG. 4a is a structural schematic diagram of the analyte detection system comprising magnetic component and magnetic induction element according to an embodiment of the invention;

FIG. 4b is a structural schematic diagram of the wake-up module of an analyte detection device comprising a magnetic induction element according to an embodiment of the invention;

FIG. 4c is a schematic diagram of the wake-up module of the analyte detection device comprising a magnetic induction element according to an embodiment of the invention;

FIG. 5a is a structural schematic diagram of the analyte detection system comprising an acceleration sensor according to an embodiment of the invention;

FIG. 5b is a structural schematic diagram of the wake-up module of the analyte detection device comprising an acceleration sensor according to an embodiment of the invention;

FIG. 5c is a functional schematic diagram of the wake-up module of the analyte detection device comprising the acceleration sensor to an embodiment of the invention;

FIG. 6 is a schematic diagram of the communication connection between the analyte detection device and remote equipment according to the embodiment of the invention;

FIG. 7 is a schematic diagram of the process of establishing communication connection between the analyte detection device and the remote equipment according to the embodiment of the invention.

### DETAILED DESCRIPTION

As mentioned above, after the analyte detection device of the prior art establishes the communication connection with the remote equipment, the higher power is used to transmit signals, so that the remote equipment can maintain the communication connection with the analyte detection device, but this kind of communication connection mode requires more energy consumption of the battery, which is not conducive to the long-term operation of the system.

In order to solve this problem, the invention provides an analyte detection device communication system, which includes an analyte detection device and remote equipment. The analyte detection device transmits signals. The remote equipment is used to search and identify nearby signals, establish the communication connection with the analyte detection device and perform data interaction. The data interaction mode can be adjusted according to the actual use. While transmitting the analyte parameter information, the battery energy consumption is reduced, which is conducive to the long-time work of the system and improves the user experience.

Various exemplary embodiments of the invention will now be described in detail with reference to the attached drawings. It is understood that, unless otherwise specified, the relative arrangement of parts and steps, numerical expressions and values described in these embodiments shall not be construed as limitations on the scope of the present invention.

In addition, it should be understood that the dimensions of the various components shown in the attached drawings are not necessarily drawn to actual proportions for ease of description, e. g. the thickness, width, length or distance of some elements may be enlarged relative to other structures.

The following descriptions of exemplary embodiments are illustrative only and do not in any sense limit the invention, its application or use. Techniques, methods and devices known to ordinary technicians in the relevant field may not be discussed in detail here, but to the extent applicable, they shall be considered as part of this manual.

It should be noted that similar labels and letters indicate similar items in the appending drawings below, so that once an item is defined or described in one of the appending drawings, there is no need to discuss it further in the subsequent appending drawings.

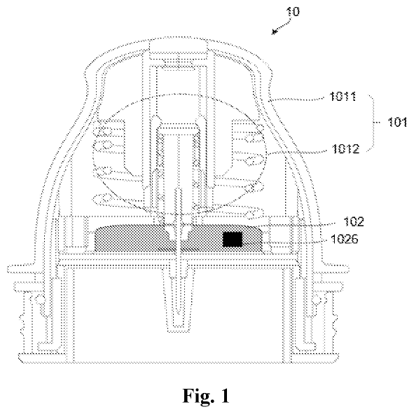

FIG. 1 is a structural diagram of the analyte detection system in an embodiment of the invention. The analyte detection system 10 comprises an auxiliary installer 101 and an analyte detection device 102. The auxiliary installer 101 comprises a housing 1011 and an auxiliary mounting module 1012, which is located inside the housing 1011. Analyte detection device 102 is located at the ejector end of auxiliary mounting module 1012, which enables rapid installation of analyte detection device 102 to the host skin surface when in use.

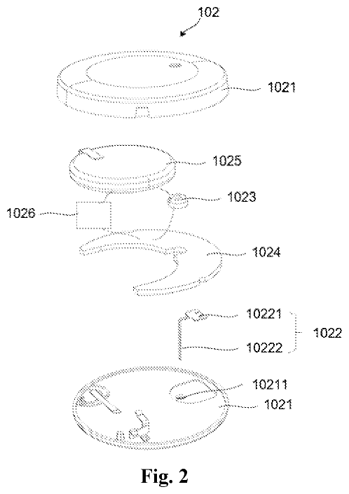

FIG. 2 is a schematic diagram of the analyte detection device to an embodiment of the invention. The analyte detection device 102 comprises the shell 1021, the sensor 1022, the transmitter 1023, the internal circuit 1024, the battery 1025 and the wake-up module 1026. Sensor 1022 comprises an external part 10221 and an internal part 10222. The external part 10221, transmitter 1023, internal circuit 1024, battery 1025 and wake-up module 1026 are located inside the shell 1021. The internal part 10222 passes through the through-via 10211 on the shell 1021 to the outside to puncture the host subcutaneous and detect the parameter information of analyte. What technicians in this field can know is that in order to pierce the internal part 10222 subcutaneous to the host, the through-via 10211 is located on the side of shell 1021 which is away from housing 1011, and at the same time, a tape (not shown in the figure) is arranged on the surface, which is used to attach the analyte detection device 102 to the skin surface of the host. The external part 10221 is electrically connected with the transmitter 1023 through the internal circuit 1024, which can transmit analyte parameter information to the outside equipment.

Before use, the shell 1021 of the analyte detection device 102 is releasable connected with the housing 1011 of auxiliary mounting device 101. Here, “releasable connection” means that shell 1021 is connected with housing 1011 by means of buckle, clamp, etc. Under the action of ejector mechanism of auxiliary mounting module 1012, the shell 1021 can be separated from housing 1011.

After the life of the sensor 1022 has expired, or the battery 1025 has run out of power, or other factors have caused the analyte detection device to fail, the user removes the entire analyte detection device from the skin surface of the host, discards it and replaces it with a new analyte detection device, is beneficial to maintain the best use of the parts of the device.

When analyte detection device 102 is installed on the skin surface of the host and starts to use, communication needs to be established with outside equipment such as PDM (Personal Diabetes Manager), mobile phone, etc., for data interaction, so as to transmit the detected analyte information data in the host to outside equipment.

As mentioned above, the analyte detection device 102 is in dormant state and transmits signal to the outside equipment at the first frequency until communication is formally established with the outside equipment. In the embodiment of the invention, the analyte detection device 102 transmits signal at a lower first frequency to an outside equipment in dormant state to reduce battery energy consumption. In the more preferred embodiment of the invention, the first frequency is 0˜12 times/hour. In the more preferred embodiment of the invention, the first frequency is 0 times/hour, that is, the analyte detection device 102 does not transmit signal to the outside equipment in dormant state.

In order to establish communication between the analyte detection device 102 which is in dormant state and outside equipment, wake-up module 1026 wakes up analyte detection device 102 according to triggering conditions, so that it enters the working state and transmits signal to the outside equipment with the second frequency, and then communication is established after the outside equipment responds. The second frequency is higher than the first frequency in order to obtain analyte parameter information conveniently and in real time. In the preferred embodiment of the invention, the second frequency is 12˜3600 times/hour. In a more preferred embodiment of the invention, the second frequency is 30 times/hour.

### First Embodiment

### Light Sensing Element

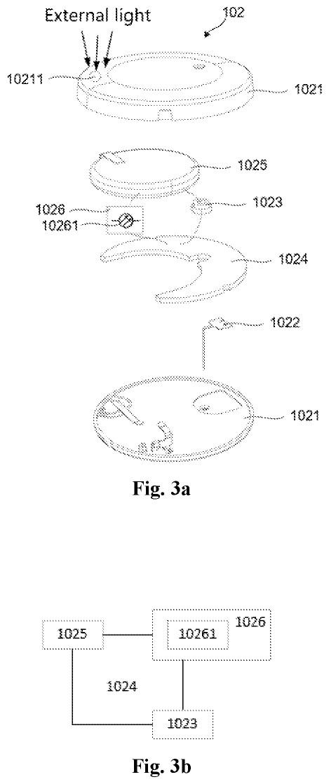

FIG. 3a is a schematic diagram of the structure of the wake-up module of the analyte detection device comprising a light sensing element in an embodiment of the invention. FIG. 3b is a functional schematic diagram of the wake-up module of the analyte detection device comprising the light sensing element in an embodiment of the invention.

In the embodiment of the invention, the wake-up module 1026 comprises a light sensing element 10261, such as photoelectric switch, which is in open state when there is no light beam or weak light beam irradiation and in a closed state when there is light beam irradiation.

In combination with FIG. 1 and FIG. 3b, transmitter 1023 is connected with battery 1025 through internal circuit 1024, forming a closed loop. The circuit is connected with a wake-up module 1026, which is connected with a light sensing element 10261 inside. The triggering condition of the wake-up module 1026 is the light intensity change received by the light sensing element 10261. In the preferred embodiment of the invention, the triggering condition of the wake-up module 1026 is that the light intensity received by the light sensing element 10261 changes from weak to strong.

In the embodiment of the invention, the analyte detection device 102 is not separated from the auxiliary mounting device 101 before it is installed on the skin surface of the host, and the shell 1021 and housing 1011 form a closed and opaque space. Since the light-transmitting area 10211 is located near the end of the housing 1011, there is no external light irradiates on light sensing element 10261, battery 1025 supplies power to transmitter 1023 through wake-up module 1026 (comprising light sensing element 10261), light sensing element 10261 is in open state, and thus the transmitter 1023 is in dormant state, and analyte detection device 102 transmits signal to outside equipment at the first frequency. After the analyte detection device 102 is installed on the skin surface of the host through the auxiliary mounting module 1012, the shell 1021 is separated from the housing 1011, and the external light can be irradiated to the light sensing element 10261 through the shell 1021. The light sensing element 10261 is in closed state. The transmitter 1023 enters the working state, and the analyte detection device 102 transmits signal to the outside equipment at the second frequency. After the response of the outside equipment, the communication is established and the analyte detection data is transmitted to the outside equipment.

In the embodiment of the invention, the shell 1021 is made of light transmittance material, such as one of polymethyl methacrylate (PMMA), polystyrene (PS), polycarbonate (PC) or poly 4-methyl-1-pentene (TPX), and the light transmittance of these material is 40%˜95%. After the separation of shell 1021 and housing 1011, the external light can be irradiated on the light sensing element 10261 through the shell 1021.

In other embodiment of the invention, the shell 1021 comprises light-transmitting area 10211, the light transmittance of the light-transmitting area 10211 is higher than that of the shell 1021, so that more external light is irradiated on the light sensing element 10261, the light intensity variation of the light sensing element 10261 is increased, and the reliability of the light sensing element 10261 is improved.

In another embodiment of the invention, the light-transmitting area 10211 comprises at least one light-transmitting hole, or an array combination of several light-transmitting holes. The light-transmitting hole can make more external light illuminate on the light sensing element 10261, further increase the light intensity variation of the light sensing element 10261, and improve the reliability of the light sensing element 10261. A light-transmittance film is arranged in the light-transmitting hole (not shown in the figure out), which can prevent external water droplets, dust and other dirt from inputting the analyte detection device through the light-transmitting hole and improve the reliability of the device.

In the embodiment of the invention, the light sensing element 10261 can sense visible light or invisible light, such as infrared or ultraviolet light. In the preferred embodiment of the invention, the light sensing element 10261 senses visible light so that the user can wake up the analyte detection device indoors or outdoors.

In other embodiment of the invention, the switch condition of open circuit and closed circuit of the light sensing element is low light irradiation to strong light irradiation, that is, before the separation of shell 1021 and housing 1011, weak external light is allowed to illuminate the interior of housing 1011, and the light sensing element 10261 receives weak light, but it is still in open circuit and the transmitter 1023 is in dormant state, which takes into account that the actual connection between shell 1021 and housing 1011 is not completely sealed. When the shell 1021 is separated from the housing 1011, the external light completely irradiates on the light sensing element 10261 through the shell 1021, and the light intensity received by the light sensing element 10261 becomes stronger. After reaching the set light intensity threshold, the light sensing element 10261 switches to the closed state, and the transmitter 1023 enters the working state to transmit signal to the outside equipment at the second frequency. After the response from the outside equipment, the communication is established and the analyte detection data is transmitted to the outside equipment.

### Second Embodiment

### Magnetic Component and Magnetic Induction Element

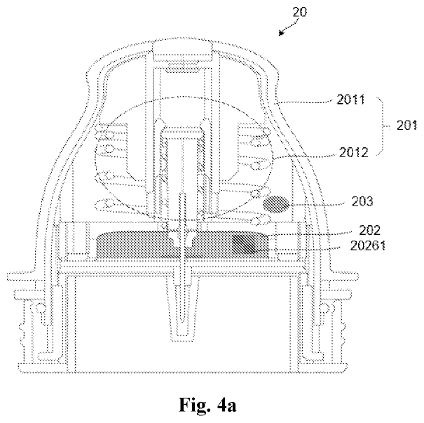

FIG. 4a is a schematic diagram of the structure of the analyte detection system comprising magnetic component and magnetic induction element in an embodiment of the invention. FIG. 4b is a schematic diagram of the structure of the wake-up module of the analyte detection device comprising the magnetic induction element in an embodiment of the invention. FIG. 4c is a schematic diagram of the function of the wake-up module of the analyte detection device comprising the magnetic induction element in an embodiment of the invention.

In the embodiment of the invention, a magnetic component 203 is arranged on the housing 2011, and a magnetic induction element 20261 is arranged in the wake-up module 2026, the battery 2025 supplies power to transmitter 2023 through the wake-up module 2026 (comprising the magnetic induction element 20261). Magnetic component 203 provides a stable magnetic field, and magnetic induction element 20261 is located in the magnetic field of magnetic component 203 and induces the magnetic field of magnetic component 203 to generate a signal. The triggering condition of the wake-up module 2026 is the magnetic field change induced by the magnetic induction element 20261.

The transmitter 2023 is connected with the battery 2025 through the internal circuit 2024, forming a closed loop, and the circuit is connected with the wake-up module 2026. Before the analyte detection device 202 is installed on the skin surface of the host, the analyte detection device 202 is not separated from the auxiliary mounting device 201, and the relative position is fixed. The magnetic field induced by the magnetic induction element 20261 to the magnetic component 203 is stable. Under the stable magnetic field, the magnetic induction element 20261 is in the open state, the transmitter 2023 is in dormant state, and analyte detection device 202 transmits signal to outside equipment at the first frequency. After the analyte detection device 202 is installed on the skin surface of the host through the auxiliary mounting module 2012, the shell 2021 is separated from the housing 2011, and the distance between the magnetic induction element 20261 and the magnetic component 203 changes, so the induced magnetic field also changes, and the magnetic induction element 20261 switches to the closed state, and transmitter 2023 enters the working state. Analyte detection device 202 transmits signal to the outside equipment at the second frequency, and then establishes communication with outside equipment after the response of the outside equipment, and transmits analyte detection data to the outside equipment.

In the embodiment of the invention, the magnetic induction element 20261 senses the magnetic field strength or magnetic field direction of the magnetic component 203. Preferably, the induction element 20261 comprises a hall element (not shown in the figure out) that sensitively sensitizes the magnetic field strength of the magnetic component 203.

In the embodiment of the invention, the magnetic component 203 may be an individual part independent of the housing 2011, or a part of the housing 2011 which is embedded in the housing 2011.

In other embodiments of the invention, the housing 2011 is embedded or enclosed with a magnetic field shielding device (not shown in the figure out), such as a Faraday cage. Technicians in this field can know that the magnetic shielding device is located outside the magnetic component 203 to reduce the impact of external magnetic field on the magnetic induction element 20261.

### Third Embodiment

### Acceleration Sensor

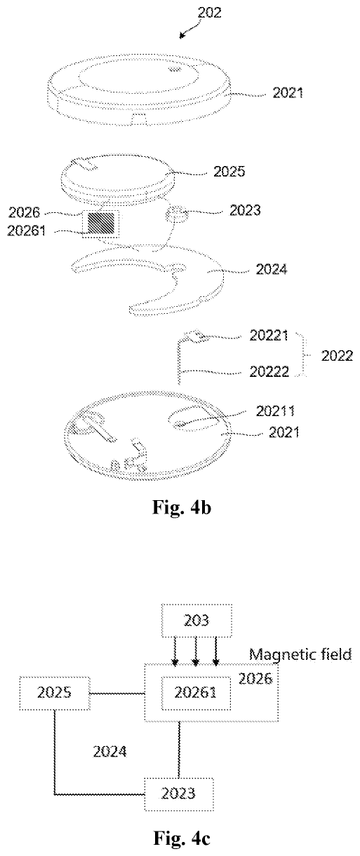

FIG. 5a is a schematic diagram of the structure of the wake-up module of the analyte detection system comprising the acceleration sensor in an embodiment of the invention. FIG. 5b is a schematic diagram of the structure of the wake-up module of the analyte detection device comprising the acceleration sensor in an embodiment of the invention. FIG. 5c is a schematic diagram of the function of the wake-up module of the analyte detection device comprising the acceleration sensor in an embodiment of the invention.

In the embodiment of the invention, the wake-up module 3026 comprises an acceleration sensor 30261, which can sensitively sense the values of motion parameters such as acceleration and adjust the circuit state of the wake-up module 3026 accordingly. The triggering condition of wake-up module 3026 is the motion parameter change of acceleration sensor 30261.

Transmitter 3023 is connected with battery 3025 through internal circuit 3024 to form a closed loop, and the circuit is connected with the wake-up module 3026, the battery 3025 supplies power to transmitter 3023 through wake-up module 3026 (comprising acceleration sensor 30261). Before the analyte detection device 302 is installed on the skin surface of the host, the analyte detection device 302 and the auxiliary mounting device 301 are relatively fixed. In order to pierce the internal part of the sensor 30222 of the analyte detection device into the skin of the host and reduce the pain sensation during the stabbing, the auxiliary mounting module 3012 adopts ejector mechanism 30121. Such as spring and other elastic parts, through the auxiliary needle 30122 can quickly pierce the body part 30222 into the host subcutaneous. When the ejector mechanism 30121 is in use, it produces a large instantaneous forward acceleration a1, and when it is installed on the skin surface of the host, it is obstructed by the skin to produce a reverse acceleration a2. After the acceleration sensor 30261 senses the above two accelerations, it can be determined that the analyte detection device 302 is installed on the skin surface of the host.

In the embodiment of the invention, before the analyte detection device 302 is installed on the skin surface of the host, the wake-up module 3026 is in an open state, and the transmitter 3023 is in a dormant state and transmits signal to the outside equipment at the first frequency. Acceleration sensor 30261 determines that the analyte detection device 302 is installed on the skin surface of the host, and the wake-up module 3026 switches to the closed state, and transmitter 3023 enters the working state and transmits signal to the outside equipment at the second frequency. After the response of the outside equipment, the communication is established and the analyte detection data is transmitted to the outside equipment.

It can be understood by those skilled in the art that “first frequency” and “second frequency” in this patent refer to the transmission frequency of the signal to characterize the transmission interval of the signal.

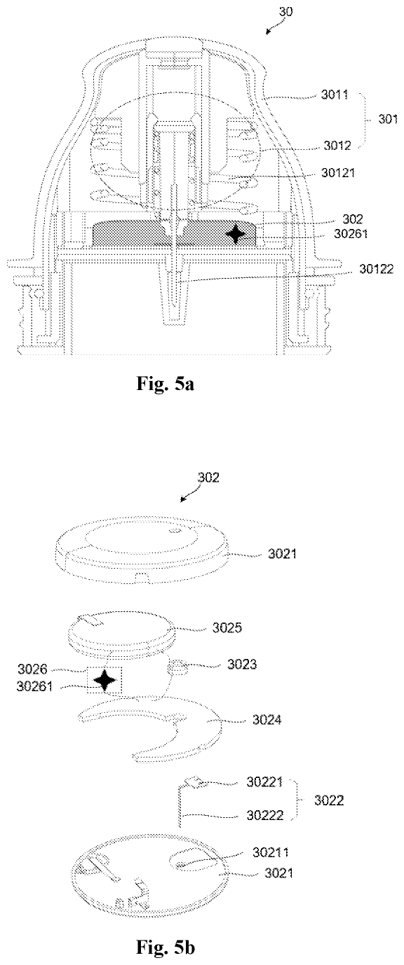

FIG. 6 is a schematic diagram of the communication connection between the analyte detection device and remote equipment according to the embodiment of the present invention.

In the embodiment of the invention, the analyte detection device 102 is activated after inputting the working state, transmits the first signal before establishing communication with the remote equipment 103, and transmits the second signal after establishing communication with the remote equipment 103.

In some embodiments of the invention, the first signal uses low-power Bluetooth and the second signal uses near-field communication (NFC). Or the first signal uses WiFi and the second signal uses low-power Bluetooth.

In other embodiments of the invention, the first signal and the second signal are of the same type, but their signal strengths are different. For example, the signal strength of the first signal is weaker than that of the second signal. In the preferred embodiment of the invention, the strengths of the first signal and the second signal are set so that the effective range of the first signal is 0˜10 m and the effective range of the second signal is 0˜10 m. In the more preferred embodiment of the invention, the effective range of the first signal is 0˜1m , and the smaller effective range of the first signal is convenient for the remote equipment 103 to filter the fault first signal.

In other embodiments of the invention, the first signal and the second signal have different signal formats, for example, the communication connection status of the first signal packet is marked as A, and the communication connection status of the second signal packet is marked as B. The above mark can be located at any position of the packet, such as the packet header or the packet body, and so on. In a preferred embodiment of the invention, the packet header is set as the communication connection status flag bit. When the communication connection is not established, the first signal packet sent by the analyte detection device is A⋅⋅⋅ while the communication connection is established, the second signal packet sent by the analyte detection device is B⋅⋅⋅. In another preferred embodiment of the invention, a plurality of flag bits are set as communication connection status mark. In the state of no communication connection, the first signal packet sent by the analyte detection device is A⋅⋅⋅A⋅⋅⋅A⋅⋅⋅, and in the state of established communication connection, the second signal packet sent by the analyte detection device is B⋅⋅⋅B⋅⋅⋅B⋅⋅⋅. As long as the communication connection status of the analyte detection device can be distinguished, the number of flag bits and format of the data packet are not limited. In this embodiment, the form of the communication connection status mark A (B) in the data packet can be a single byte, such as 0 (1), or a multi byte, such as 000 (111), which is not limited here.

In other embodiments of the invention, the first signal and the second signal have different signal frequencies, for example, the first signal is the low-frequency signal and the second signal is the high-frequency signal, or the first signal is the high-frequency signal and the second signal is the low-frequency signal. Wherein, the first signal is the low-frequency signal and the second signal is the high-frequency signal, which is only used to explain that the frequency of the first signal is lower than the second signal, rather than to limit the specific frequency of the first signal and the second signal. Similarly, the first signal is the high-frequency signal and the second signal is the low-frequency signal, which is only used to explain that the frequency of the first signal is higher than the second signal, rather than to limit the specific frequency of the first signal and the second signal.

In other embodiments of the invention, the difference between the first signal and the second signal lies in the signal format and signal strength, or in the signal format and signal strength, the signal frequency, or a combination of other signal difference forms. The combination of various different forms of signals is more conducive to the remote equipment to distinguish the analyte detection device to be connected.

As long as the first signal can be distinguished from the second signal, the technical scheme comprising but not limited to the above can be adopted, which is not limited here. No matter how the first signal and the second signal are distinguished, the characteristics of the first signal and the second signal are pre stored in the remote equipment 103.

In the embodiment of the invention, the second signal also includes at least the scanning signal and the real-time signal, that is, after the analyte detection device 102 establishes communication connection with the remote equipment 103, they execute a data interaction program. Here, the data interaction program can be that the analyte detection device 102 sends the real-time in vivo analyte parameter information to the remote equipment 103 at a fixed time interval, or when the remote equipment 103 is close to the analyte detection device 102, the communication connection with the remote equipment 103 is re-established. At this time, the analyte detection device 102 sends the in vivo analyte parameter information to the remote equipment 103. The specific scheme will be described in detail below.

In the embodiment of the invention, after the remote equipment 103 establishes communication connection with the analyte detection device 102, the user can select a scanning program or a real-time program in the remote equipment 103.

For example, when the user needs to monitor the blood glucose information in the body in real time during the day when he/she has a normal work, rest and diet, the user selects a real-time program, and the analyte detection device 102 sends the blood glucose concentration information in the body to the remote equipment 103 at a fixed time interval for the user, so that the user can adjust the work, rest and diet. In the preferred embodiment of the invention, the analyte detection device 102 sends in vivo blood glucose concentration information to the remote equipment 103 at a time interval of 2 min.

For another example, when the user cannot know the real-time blood glucose concentration information after entering sleep at night, the user selects the scanning program. During sleep, the analyte detection device 102 does not send the blood glucose concentration information to the remote equipment 103. After the user wakes up, the remote equipment 103 is close to the analyte detection device 102, and the analyte detection device 102 sends the blood glucose concentration information to the remote equipment 103. Generally, after the user selects the scanning program, the effective distance of the signal sent by the analyte detection device 102 is very short, for example, 0˜1 m, or even 0˜0.1 m, or 0˜0.01 m. A shorter effective distance of the signal can prevent the user from misoperation and improve the user experience.

In the embodiment of the invention, the user completes the switching between the scanning program and the real-time program by inputting or adjusting the control command on the remote equipment 103. When switching the data interaction program, the remote equipment 103 has established the communication connection with the analyte detection device 102.

In the embodiment of the invention, the power of the analyte detection device 102 transmitting the scanning signal is lower than the power of the real-time signal, so that the effective distance of the scanning signal is less than the effective distance of the real-time signal. When the user uses the real-time program, the remote equipment 103 is held in his hand or placed on the table, and will not deliberately close to the analyte detection device 102 installed on his arm or waist, so the two signals will not interfere, the reliability of real-time signal is guaranteed. In the preferred embodiment of the invention, the effective distance of the real-time signal is 0˜10 m to ensure that the user can maintain the communication connection between the remote equipment 103 and the analyte detection device 102 during daily use.

In the embodiment of the invention, the power of the real-time signal or the scanning signal can be adjusted according to the communication distance of the signal. For example, when using the real-time program, it is not convenient for the user to carry the remote equipment 103 when bathing, but the remote equipment 103 needs to be placed away from the bathing place, or even in another room. At this time, it is necessary to appropriately increase the power of the signal to increase the effective distance of the signal, so that the remote equipment 103 can maintain the communication connection with the analyte detection device 102. After bathing, the user can hold the remote equipment 103, at this time, the power of the signal can be reduced. While the remote equipment 103 maintains the communication connection with the analyte detection device 102, the energy consumption of the battery can be reduced. In the embodiment of the invention, the adjustment of signal power can be manually adjusted by the user or automatically adjusted by the analyte detection device 102.

In the embodiment of the invention, when the remote equipment 103 is within the effective distance of the signal, the remote equipment 103 establishes the communication connection with the analyte detection device 102, and when the remote equipment 103 is outside the effective distance of the signal, the remote equipment 103 disconnects the communication connection with the analyte detection device 102. For example, when the user uses the scanning program, the remote equipment 103 is located outside the effective distance of the signal, and the remote equipment 103 disconnects the communication connection with the analyte detection device 102. At this time, there is no data interaction between the remote equipment 103 and the analyte detection device 102, which can reduce the energy consumption of the battery. When the remote equipment 103 enters the effective distance of the signal, the remote equipment 103 re-establishes the communication connection with the analyte detection device 102. For another example, when the user uses the real-time program, when the user exercises, the remote equipment 103 will generally not be carried with him, and the remote equipment 103 will maintain a long distance from the analyte detection device 102. At this time, the remote equipment 103 disconnects the communication connection with the analyte detection device 102. After exercising, the remote equipment 103 will be carried, and the remote equipment 103 will enter the effective distance of the signal, the remote equipment 103 re-establishes the communication connection with the analyte detection device 102.

In the embodiment of the invention, when the user uses the scanning program, in the normal time, the remote equipment 103 and the analyte detection device 102 are in the disconnected communication connection state. When the user operates the remote equipment 103 to approach the analyte detection device 102 every time, for example, 4 cm, the remote equipment 103 searches for the signal transmitted by the analyte detection device 102 and re-establishes the communication connection with the analyte detection device 102. The analyte detection device 102 interacts with the remote equipment 103 and sends the in vivo analyte parameter information to the remote equipment 103. Here, the analyte detection device 102 can interact with the remote equipment 103 once or many times. Generally, when the user selects to use the scanning program, the time for the remote equipment 103 to approach the analyte detection device 102 is very short. Therefore, it is preferred that the analyte detection device 102 conduct a data interaction with the remote equipment 103. When the analyte detection device 102 interacts with the remote equipment 103, it can transmit historical analyte parameter information, such as the data set in the past hour, or real-time analyte parameter information, or historical analyte parameter information with real-time analyte parameter information at the same time, which is not limited here. In the embodiment of the invention, when the user uses the real-time program, in the normal time, the remote equipment 103 and the analyte detection device 102 are in the communication connection state, and the analyte detection device 102 sends the analyte parameter information to the remote equipment 103 at a fixed time interval, such as 2 min. The specific time interval is not specifically limited here.

In the embodiment of the invention, the effective distance of the scanning signal is less than the effective distance of the real-time signal. Generally, the two signals will not interfere, but in some complex environments, such as in the hospital ward, multiple patients use the analyte detection device, resulting in the presence of multiple scanning signals or real-time signals in a narrow space, and there is a low probability of signal interference, which is detrimental to patients. To avoid this, scanning signals and real-time signals can also be distinguished by signal frequency, signal type or/and signal format. In a preferred embodiment of the invention, the scanning signal and the real-time signal have different signal formats. For example, the mark of the scanning signal packet is C, and the mark of the real-time signal packet is D. The above mark can be located at any position of the data packet, such as the packet header, or the packet body, and so on. In a preferred embodiment of the invention, the packet header of the data packet is set as a marker bit to distinguish between the scanning signal and the real-time signal. In another preferred embodiment of the invention, a plurality of marker bits of the data packet are set as the marker bits to distinguish the scanning signal and the real-time signal. When using the scanning program, the scanning signal data packet sent by the analyte detection device is C⋅⋅⋅C⋅⋅⋅C⋅⋅⋅. And when using the real-time program, the real-time signal data packet sent by the analyte detection device is D⋅⋅⋅D⋅⋅⋅D⋅⋅⋅. As long as the scanning signal and the real-time signal can be distinguished, the number of tag bits and format of the data packet are not limited. In this embodiment, the form of scanning signal and real-time signal distinguishing mark C (D) in the data packet can be single byte, such as 0 (1), or multi byte, such as 000 (111), which is not limited here.

In the embodiment of the invention, the analyte detection device 102 may re-establish the communication connection with the remote equipment 103 many times within its service life. In some complex environments, such as in the hospital ward, the signals between multiple devices may be disturbed, and the user may receive error signals from other devices when preparing to establish the communication connection between the remote equipment 103 and the analyte detection device 102, In order to avoid this possible situation, before the remote equipment 103 establishes the communication connection with the analyte detection device 102, the remote equipment 103 sends a prompt that needs to be confirmed by the user. For example, the device code and other features of the analyte detection device 102 that is about to establish the communication connection are displayed on the remote equipment, and the communication connection can be formally established only after the user confirms.

In the embodiment of the invention, once the analyte detection device 102 establishes the communication connection with the remote equipment 103 for the first time, the analyte detection device 102 is stored as a white list device of the remote equipment 103, and its signal characteristics are recorded by the remote equipment 103 at the same time. When searching for nearby signals, the remote equipment 103 can only recognize the signals transmitted by the recorded white list device and establish the communication connection with the white list device, which can avoid the interference of other device signals and improve the reliability of the communication connection.

In other embodiments of the invention, the analyte detection device 102 can simultaneously transmit scanning signals and real-time signals. For example, in the hospital, on the one hand, the patient's blood glucose concentration information needs to be transmitted to the doctor's medical management system (remote equipment) to provide doctors with real-time treatment basis. At this time, the analyte detection device 102 needs to interact with the medical management system in real time. On the other hand, the patient also needs to know the blood glucose concentration information in the body when needed, at this time, the patient can get the blood glucose concentration information by putting his PDM or mobile phone close to the analyte detection device 102 on his body to receive the scanning signal.

In the embodiment of the invention, after the remote equipment 103 searches the signal, before establishing the communication connection with the analyte detection device 102, it is necessary to execute a communication data interaction program to match the analyte detection device 102 with the remote equipment 103. The communication data interaction program needs to last for a period of time, and the duration is too short, which is easy to cause user misoperation. For example, some users will install an analyte detection device 102 on their arms and bellies in order to monitor the blood glucose concentration information in their bodies more accurately. Each analyte detection device 102 needs to be connected to the remote equipment 103 to transmit data, so when the user operates to establish the communication connection, it is possible to approach the remote equipment 103 to the wrong analyte detection device 102. For example, the user holds the remote equipment 103 ready to establish the communication connection with the analyte detection device 102 installed on the arm, and when raising his hand, he passes through the analyte detection device 102 installed on the belly, which will establish the wrong communication connection. In this case, by setting the minimum effective time condition for the remote equipment 103 to be close to the analyte detection device 102, such as 2 s, the wrong communication connection caused by misoperation can be avoided. Even if the remote equipment 103 is close to the wrong analyte detection device 102 due to misoperation, it cannot establish the communication connection with the wrong analyte detection device 102 due to insufficient duration, which improves the reliability of the communication connection. If the communication data interaction program lasts too long, on the one hand, it will increase the energy consumption of the battery, on the other hand, the user waiting time is too long, which will affect the user experience. Therefore, it is also necessary to set the maximum effective time condition for the remote equipment 103 to be close to the analyte detection device 102, such as 7 s. That is, when the remote equipment 103 is close to the analyte detection device 102 for not less than 2 s, but not more than 7 s, the remote equipment 103 can normally establish the communication connection with the analyte detection device 102, otherwise it cannot normally establish the communication connection. Those skilled in the art can know that the effective time conditions here are not limited to the time limits described here, but can be adjusted according to the actual product performance, user needs and other conditions.

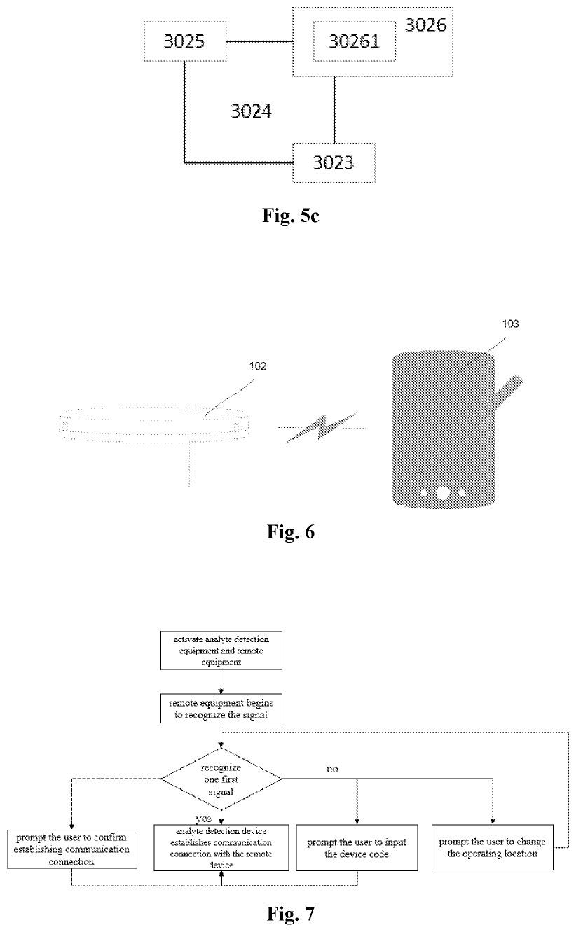

FIG. 7 is a schematic diagram of the process of establishing communication connection between the analyte detection device and the remote equipment in an embodiment of the invention. Refer to the FIG. 7, in the embodiment of the invention, when the user needs to establish the communication connection between the analyte detection device 102 and the remote equipment 103, first place the remote equipment 103 near the analyte detection device 102 and confirm that there are no other analyte detection devices around as far as possible. After the user starts the remote equipment 103, the remote equipment 103 searches and identifies the nearby signal. If the remote equipment 103 recognizes only one first signal, it can be judged that the signal is the signal transmitted by the analyte detection device 102 to establish the communication connection, and the communication connection with the analyte detection device 102 is established through the link of the signal, without the user manually inputting or scanning the equipment code of the analyte detection device 102, it simplifies the process of establishing communication connection, avoids users from inputting or scanning wrong device code, and improves the user experience.

In other embodiments of the invention, before the analyte detection device 102 establishes the communication connection with the remote equipment 103, the remote equipment 103 prompts the user to confirm whether to connect, so as to improve the reliability of the communication connection between the analyte detection device 102 and the remote equipment 103.

In the embodiment of the invention, if the user is in a complex environment, that is, the remote equipment 103 recognizes multiple first signals, or multiple first signals and second signals, and the remote equipment 103 cannot judge the first signal transmitted by the analyte detection device 102 to establish the communication connection, in this case, the remote equipment 103 prompts the user to manually input or scan the device code of the analyte detection device 102 to establish the communication connection, In order to establish the communication connection with the analyte detection device 102.

In other embodiments of the invention, when the user is in a complex environment, the remote equipment 103 cannot judge the first signal transmitted by the analyte detection device 102 to establish the communication connection, and the remote equipment 103 prompts the user to change the operation location. The user needs to carry the analyte detection device 102 and the remote equipment 103 to other locations until the remote equipment 103 recognizes only one first signal, It can be judged that the signal is the signal transmitted by the analyte detection device 102 to establish the communication connection, and establish the communication connection with the analyte detection device 102 through the link of the signal, without the user manually inputting or scanning the equipment code of the analyte detection device 102, which simplifies the process of establishing the communication connection, avoids the user inputting or scanning the wrong equipment code, and improves the user experience.

In other embodiments of the invention, if the remote equipment 103 does not recognize the first signal within the effective range, it is judged that the analyte detection device 102 is not working normally. At this time, the remote equipment 103 sends an alarm or fault prompt to the user to prompt the user to check or replace the analyte detection device.

In the embodiment of the invention, the prompt of the remote equipment 103 may be one or more of the forms of audio, video or vibration. In one embodiment of the invention, when the prompt of the remote equipment 103 is audio, according to different prompt needs, the remote equipment 103 sends out “tick” prompt tones with different lengths and/or time intervals. In another embodiment of the invention, when the prompt of the remote equipment 103 is video, different text prompts are displayed on the display screen according to different prompt needs. In another embodiment of the invention, when the prompt of the remote equipment 103 is vibration, the remote equipment 103 vibrates with different lengths and/or time intervals according to different prompt needs.

To sum up, the embodiment of the invention discloses an analyte detection device communication system, which comprises the analyte detection device and the remote equipment. The analyte detection device transmits signals. The remote equipment is used to search and identify nearby signals, establish the communication connection with the analyte detection device and perform data interaction. The data interaction mode can be adjusted according to the actual use. While transmitting the analyte parameter information, the battery energy consumption is reduced, which is conducive to the long-time work of the system and improves the user experience.

Although some specific embodiments of the invention have been detailed through examples, technicians in the field should understand that the above examples are for illustrative purposes only and are not intended to limit the scope of the invention. Persons skilled in the field should understand that the above embodiments may be modified without departing from the scope and spirit of the present invention. The scope of the invention is limited by the attached claims.

1. A communication system of analyte detection device is characterized by, comprising:
an analyte detection device, which is installed on a surface of a user's skin, is configured to obtain an analyte parameter information in the user's body, and transmit signals to a remote equipment; and
the remote equipment, which is used to search and identify nearby signals, is configured to establish a communication connection with the analyte detection device, and is configured to execute a data interaction program, wherein the data interaction program at least includes a scanning program and a real-time program, wherein when the scanning program is executed, the analyte detection device transmits the scanning signal with a first power, wherein when the real-time program is executed, the analyte detection device transmits a real-time signal with a second power, wherein the first power is lower than the second power.

2. Communication system of analyte detection device of claim 1, wherein the first power and the second power are adjusted according to a communication distance of the signal to adjust an effective distance of the scanning signal or the real-time signal.

3. Communication system of analyte detection device of claim 2, wherein the effective distance of the scanning signal is 0˜1 m, and the effective distance of the real-time signal is 0˜10 m.

4. Communication system of analyte detection device of claim 3, wherein when the remote equipment is within the effective distance, the remote equipment establishes the communication connection with the analyte detection device, otherwise, the communication connection with the analyte detection device is not established.

5. Communication system of analyte detection device of claim 1, wherein when the scanning program is executed, the analyte detection device sends the analyte parameter information to the remote equipment at least once.

6. Communication system of analyte detection device of claim 5, wherein the analyte parameter information includes a real-time analyte parameter information and/or a historical analyte parameter information.

7. Communication system of analyte detection device of claim 1, wherein when the real-time program is executed, the analyte detection device sends a real-time analyte parameter information to the remote equipment at a fixed time interval.

8. Communication system of analyte detection device of claim 1, wherein the difference between the scanning signal and the real-time signal also lies at least in a signal frequency, a signal type or/and a signal format.

9. Communication system of analyte detection device of claim 1, wherein before the communication connection between the remote equipment and the analyte detection device is established, the remote equipment sends a prompt that needs to be confirmed by the user.

10. Communication system of analyte detection device of claim 1, wherein after the remote equipment establishes the communication connection with the analyte detection device, the analyte detection device is stored as a white list device of the remote equipment, and the remote equipment only receives the signals transmitted by the white list device.

11. Communication system of analyte detection device of claim 1, wherein the scanning program and the real-time program are switched by a control command.

12. Communication system of analyte detection device of claim 1, wherein after the remote equipment searches the signals and before establishes communication with the analyte detection device, the remote equipment executes a communication data interaction program with the analyte detection device, and an effective time of the communication data interaction program is preset as t.

13. Communication system of analyte detection device of claim 12, wherein the effective time t of the communication data interaction program is 2˜7 s.

14. Communication system of analyte detection device of claim 13, wherein when a duration T of the communication data interaction program is shorter than or longer than the effective time t, the remote equipment does not establish the communication connection with the analyte detection device.

15. Communication system of analyte detection device of claim 2, wherein after the remote equipment searches the signals and before establishes communication with the analyte detection device, the remote equipment executes a communication data interaction program with the analyte detection device, and an effective time of the communication data interaction program is preset as t.

16. Communication system of analyte detection device of claim 15, wherein the effective time t of the communication data interaction program is 2˜7 s.

17. Communication system of analyte detection device of claim 16, wherein when a duration T of the communication data interaction program is shorter than or longer than the effective time t, the remote equipment does not establish the communication connection with the analyte detection device.
